(19) United States
(12) Patent Application Publication
(10) Pub. No.: US 2014/0323885 A1
(43) Pub. Date: Oct. 30, 2014

(54) METHODS AND SYSTEMS FOR PREDICTING ACUTE HYPOTENSIVE EPISODES

(71) Applicant: GENERAL ELECTRIC COMPANY, Schenectady, NY (US)

(72) Inventors: Sahika Genc, Schenectady, NY (US); Gyemin Lee, Seoul (KR)

(73) Assignee: General Electric Company, Schenectady, NY (US)

(21) Appl. No.: 13/869,287

(22) Filed: Apr. 24, 2013

(51) Int. Cl. A61B 5/00; A61B 5/021

(52) U.S. Cl. CPC A61B 5/7267; A61B 5/7275; A61B 5/7203; A61B 5/021; A61B 5/7282
USPC 600/485; 600/481; 600/508

(57) ABSTRACT

A method is presented. The method includes determining a plurality of temporal training features corresponding to one or more hemodynamic parameters of a plurality of elected-patients based upon temporal training signals representative of the one or more hemodynamic parameters, and generating an acute hypotension prediction classifier based upon the plurality of temporal training features corresponding to the one or more hemodynamic parameters of the plurality of elected-patients, wherein the plurality of temporal training features comprises covariance between two or more of the temporal training signals corresponding to the one or more hemodynamic parameters.

### BACKGROUND

Acute hypotensive episodes (AHEs) are one of the most critical events that generally occur in intensive care units (ICUs). An acute hypotensive episode is a clinical condition typically characterized by abnormally low blood pressure values and other related values. For example, an acute hypotensive episode may occur in an interval of 30 minutes or more during which at least 90% of the mean arterial pressure (MAP) measurements of a patient are at or below 60 mmHg. Acute hypotensive episodes may occur due to a large number of causes. The causes of acute hypotensive episodes, among others, may include sepsis, myocardial infarction, cardiac arrhythmia, pulmonary embolism, hemorrhage, dehydration, anaphylaxis, medication, vasodilatory shock, or any of a wide variety of other causes. Often it may be crucial to determine the causes of the acute hypotensive episodes to administer appropriate treatment. However, when the acute hypotensive episodes are not predicted in time, the practitioners are left with insufficient time to determine the causes of the acute hypotensive episodes. Also, due to insufficient time appropriate treatment may not be administered. If an acute hypotensive episode is not promptly and appropriately treated, it may result in an irreversible organ damage and, eventually death.

### BRIEF DESCRIPTION

A method is presented. The method includes determining a plurality of temporal training features corresponding to one or more hemodynamic parameters of a plurality of elected-patients based upon temporal training signals representative of the one or more hemodynamic parameters, and generating an acute hypotension prediction classifier based upon the plurality of temporal training features corresponding to the one or more hemodynamic parameters of the plurality of elected-patients, wherein the plurality of temporal training features comprises covariance between two or more of the temporal training signals corresponding to the one or more hemodynamic parameters.

A system is presented. The system includes a processing subsystem that is configured to determine a plurality of temporal training features corresponding to one or more hemodynamic parameters of a plurality of elected-patients based upon temporal training signals representative of the one or more hemodynamic parameters, and generate an acute hypotension prediction classifier based upon the plurality of temporal training features corresponding to the one or more hemodynamic parameters of the plurality of elected-patients, wherein the plurality of temporal training features comprises covariance between two or more of the temporal training signals corresponding to the at least one hemodynamic parameter.

### DRAWINGS

These and other features and aspects of embodiments of the present invention will become better understood when the following detailed description is read with reference to the accompanying drawings in which like characters represent like parts throughout the drawings, wherein:

FIG. 1 is a block diagram of an acute hypotension prediction system, in accordance with one embodiment of the present systems;

FIG. 2 is a diagrammatic illustration of the architecture of the processing-subsystem referred to in FIG. 1, in accordance with one embodiment of the present systems;

FIG. 3a is an exemplary graphical representation of a temporal signal representative of a hemodynamic parameter, in accordance with an embodiment of the present systems;

FIG. 3b is an exemplary graphical representation of a preprocessed temporal signal, in accordance with an embodiment of the present techniques;

FIG. 4 is an exemplary block diagram that shows features corresponding to a temporal signal representative of a hemodynamic parameter namely ‘heart rate’ and another temporal signal representative of a hemodynamic parameter namely ‘Systolic arterial blood pressure’; and

FIG. 5 is a flow chart illustrating an exemplary method for generating an acute hypotension prediction classifier, in accordance with certain embodiments of present techniques.

### DETAILED DESCRIPTION

As will be described in detail hereinafter, systems and methods that predict potential acute hypotensive episodes in patients are presented. The systems and methods predict the potential acute hypotensive episodes in an automated manner without human interference. A rapid and accurate prediction of the potential acute hypotensive episodes may provide adequate time to diagnose the cause of the potential acute hypotensive episodes in the patients. Therefore, the prediction of the acute hypotensive episodes may improve possibilities of determination of the kind of intervention or treatment required to prevent the patients from the potential acute hypotensive episodes. In one embodiment, the systems and methods predict the potential acute hypotensive episodes in patients who are admitted in intensive care units (ICUs).

As used herein, a term “temporal training signal” is a time-series signal representative of a hemodynamic parameter of an elected-patient. As used herein, a term “elected-patient” refers to a patient who had or did not have AHE, and temporal training signals representative of hemodynamic parameters of the elected-patient are used to generate an acute hypotension prediction classifier. Furthermore, as used herein, a term “temporal input signal” refers to a time-series signal representative of a hemodynamic parameter of an admitted-patient. As used herein, a term “admitted-patient” refers to a patient who is monitored in real-time for prediction of a potential acute hypotensive episode in the patient.

As used herein, a term “hemodynamic parameter” refers to a cardiac parameter, a vascular parameter, an arterial parameter and a blood pressure parameter. As used herein, a term “temporal training features” are features that are determined based upon temporal training signals representative of hemodynamic parameters of a plurality of elected-patients, wherein the temporal training features are used for generating the acute hypotension prediction classifier. Furthermore, as used herein, a term “temporal input features” refers to features that are determined based upon temporal input signals representative of hemodynamic parameters of an admitted-patient, wherein the temporal input features are used for predicting an acute hypotensive episode in an admitted-patient.

In one embodiment, the present system includes a processing subsystem that generates an acute hypotension prediction classifier to predict potential acute hypotensive episodes. The processing subsystem receives temporal training signals representative of at least one hemodynamic parameter of a plurality of elected-patients. The processing subsystem determines a plurality of temporal training features based upon the temporal training signals. Furthermore, the processing subsystem generates an acute hypotension prediction classifier by applying a support vector machine technique to the plurality of temporal training features. The acute hypotension prediction classifier predicts potential acute hypotensive episode in an admitted-patient.

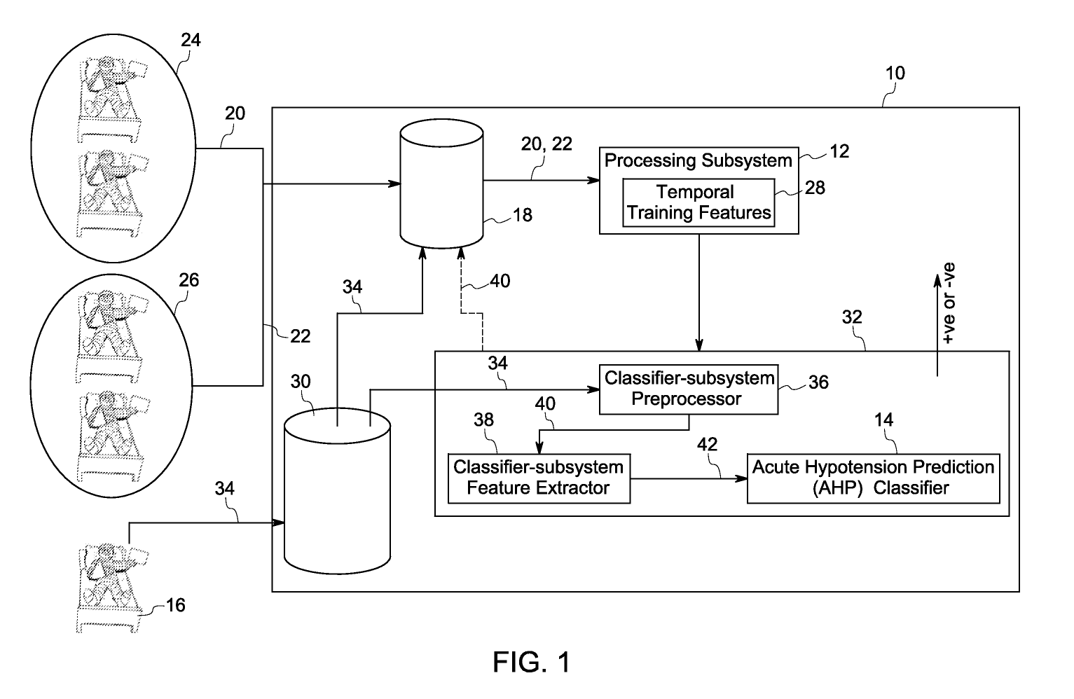

Turning now to the drawings, and referring to FIG. 1, a block diagram of an embodiment of an acute hypotension prediction system 10 is presented. The system 10 includes a processing subsystem 12 that is configured to generate an acute hypotension prediction (AHP) classifier 14. The system 10 further predicts potential acute hypotensive episodes (AHE). Particularly, the AHP classifier 14 predicts a potential acute hypotensive episode (AHE) in an admitted-patient 16. In one embodiment, the potential AHE is predicted in the admitted-patient 16 in Intensive Care Units (ICUs). However, embodiments are not limited to patients in ICUs and can be used in other settings as well. As previously noted, the term “admitted-patient” refers to a patient who is monitored in real-time for prediction of a potential AHE in the patient.

As shown in the presently contemplated configuration, the system 10 further includes a first data repository 18. The first data repository 18 stores temporal training signals 20, 22. As previously noted, the term “temporal training signal” is a time series signal representative of a hemodynamic parameter of an elected-patient. The hemodynamic parameters, for example, may include heart rate (HR), blood pressure, arterial blood pressure, diastolic arterial blood pressure (DABP), systolic arterial blood pressure (SABP), mean ambulatory blood pressure (MABP), or combinations thereof.

The temporal training signals 20, 22 are representatives of one or more hemodynamic parameters of a plurality of elected-patients 24, 26. Particularly, the temporal training signals 20, 22 are time-series measurements of the hemodynamic parameters of the elected-patients 24, 26. The temporal training signals 20 are time series measurements of at least one of the hemodynamic parameters of the elected-patients 24, and the temporal training signals 22 are time series measurements of at least one of the hemodynamic parameters of elected-patients 26. In one embodiment, a single temporal training signal represents a single hemodynamic parameter of a single elected-patient. In one embodiment, multiple temporal training signals may represent multiple and/or different heart associated parameters of a single elected-patient. For example, a temporal training signal T may represent time series measurements of a hemodynamic parameter namely ‘heart rate’ of an elected-patient E. Similarly, another temporal training signal T′ may represent time series measurements of another hemodynamic parameter namely mean Ambulatory blood pressure (MABP) of the elected patient E.

In one embodiment, the temporal training signals 20, 22 represent time-series measurements taken since admission of the elected-patients 24, 26 in the ICUs until discharge of the elected-patients 24, 26. In another embodiment, the temporal training signals 20, 22 may include time-series measurements taken during a determined time period since the admission of the elected-patients 24, 26 in the ICU/s. For example, the temporal training signals 20, 22 may be 10 or more hours' measurements of hemodynamic parameters of 60 elected-patients in intensive care unit/s. In one embodiment, the temporal training signals 20, 22 may include real-time time-series measurements of the hemodynamic parameters.

In the presently contemplated configuration, the elected-patients 24 refers to patients who had AHE and the elected-patients 26 refers to patients who did not have AHE in the past during their tenure in intensive care units ICU/s. Therefore, the elected-patients 24, 26 include patients who had or did not have acute hypotensive episodes in the past during their tenure in the ICU/s.

The first data repository 18, for example, may receive the temporal training signals 20, 22 from one or more measuring instruments/machines or diagnosis devices (not shown) that monitor or measure the hemodynamic parameters of the elected-patients 24, 26 to generate the temporal training signals 20, 22. In one embodiment, the temporal training signals 20, 22 may be sampled once per minute, for example. Other appropriate sampling times may also be used It is noted that while in the presently contemplated configuration, the temporal training signals 20, 22 are stored in the first data repository 18; in certain embodiments, the temporal training signals 20, 22 may be stored in a cloud.

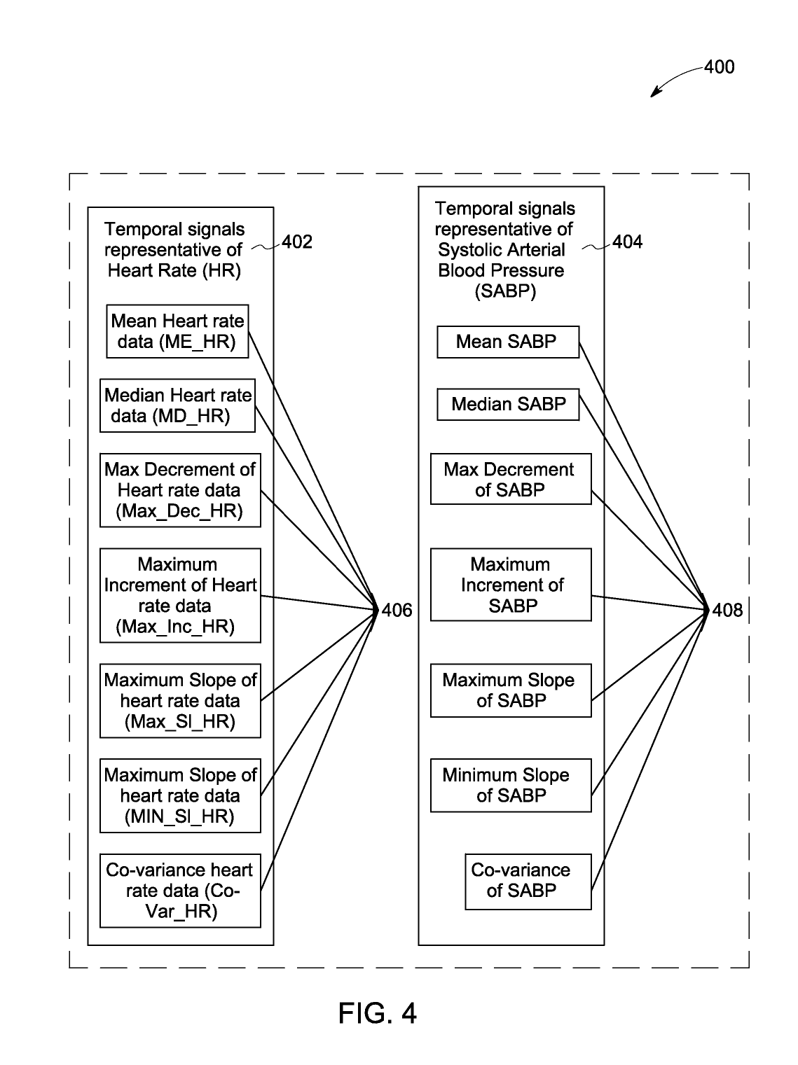

The processing subsystem 12 determines a plurality of temporal training features 28 based upon the temporal training signals 20, 22. The temporal training features 28, for example, may include covariance between two or more of the temporal training signals 20, 22 representative of the hemodynamic parameters of the elected-patients 20, 22. The temporal training features 28, for example, may further include a mean of temporal training signals, a median of temporal training signals, a maximum decrement in the expanse of temporal training signals, a maximum increment in the expanse of temporal training signals, a maximum slope of a linear regression of temporal training signals, a minimum slope of a linear regression of temporal training signals, or combinations thereof. In one embodiment, the temporal training features 28, for example, may include a single temporal training feature corresponding to each of the elected-patients 24, 26. In another embodiment, the temporal training features 28 may include multiple temporal training features corresponding to each of the elected-patients 24, 26. In one embodiment, the temporal training features 28 corresponding to each of the elected-patients 24, 26 may be same. In another embodiment, a first set of temporal training features corresponding to an elected-patient may be different from a second set temporal training features corresponding to another elected-patient. Exemplary temporal training features corresponding to a plurality of temporal training signals representative of a hemodynamic parameter namely ‘heart rate’ is shown in FIG. 4. Similarly, exemplary temporal training features corresponding to a plurality of temporal training signals representative of a hemodynamic parameter namely: ‘Systolic arterial blood pressure’ is shown in FIG. 4.

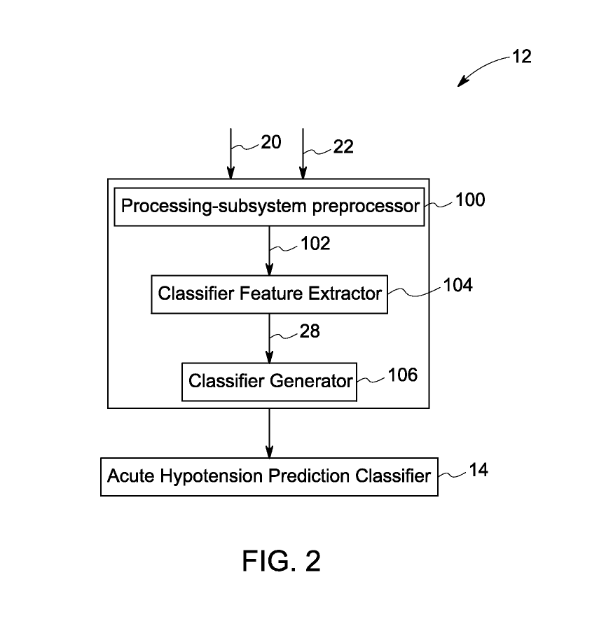

Furthermore, the processing subsystem 12 generates the acute hypotension prediction classifier 14 based upon the temporal training features 28. In one embodiment, the processing subsystem 12 generates the temporal training features 28 by applying a support vector machine technique to the temporal training features 28. The support vector machine technique, for example, may be a linear support vector machine technique. The AHP classifier 14, for example, may be a model, a hyper plane, executable instructions, or the like. The generation of the AHP classifier 14 based upon the temporal training features 28 is shown in greater detail with reference to FIG. 2.

The system 10 further includes a second data repository 30 and a classifier-subsystem 32. The second data repository 30 stores temporal input signals 34. As previously noted, the term “temporal input signal” refers to a time-series signal representative of a hemodynamic parameter of an admitted-patient. Accordingly, in this embodiment, the temporal input signals 34 are representatives of one or more hemodynamic parameters of the admitted-patient 16. Particularly, the temporal input signals 34 are time-series measurements of the hemodynamic parameters of the admitted-patient 16. For example, a temporal input signal S in the temporal input signals 34 may represent time series measurements of a hemodynamic parameter namely ‘heart rate’ of the admitted-patient 16. It is noted that the first data repository 18 may be located at a remote location from the second data repository 30. The second data repository 30, for example, may be located in a hospital, an ICU, a diagnostic center, or the like.

The second data repository 30, for example, may receive the temporal input signals 34 from one or more measuring instruments/machines or diagnosis devices (not shown) that monitor or measure the hemodynamic parameters of the admitted-patient 16 to generate the temporal input signals 34. In one embodiment, the temporal input signals 34 may be sampled once per second. However, embodiments are not limited to this sampling rate and other appropriate sampling rates may be used. It is noted that while in the presently contemplated configuration, the temporal input signals 34 are stored in the second data repository 30; in certain embodiments, the temporal input signals 34 may be stored in a cloud.

As shown in the presently contemplated configuration, the system 10 further includes the acute hypotension prediction classifier-subsystem 32. The acute hypotension prediction classifier subsystem 32 predicts the potential acute hypotension episodes (AHE) in the admitted-patient 16. Hereinafter, the term “acute hypotension prediction classifier-subsystem 32” will be referred to as “classifier-subsystem 32.” The classifier-subsystem 32 may predict the potential AHEs when the admitted-patient 16 is in an Intensive Care Unit ICU, or other location.

In one embodiment, temporal input signals 34 represent time-series measurements taken since admission of the admitted-patient 16 in the ICU until the beginning of an acute hypotension prediction time-period window. For example, if the acute hypotension prediction window starts at a time T₀, then the temporal input signals may be taken since the admission of the admitted-patient 16 in the ICU till the time T₀. In another embodiment, the temporal input signals 34 represent time-series measurements taken for a determined time period starting after the admission of the admitted-patient 16 in the ICU until the beginning of the acute hypotension prediction time-period window T₀. For example, if the acute hypotension prediction window starts at a time T₀, then the temporal input signals 34 measurements may be taken for T₀−10 hours after the admission of the admitted patient 16 in the ICU.

As shown in the presently contemplated configuration, the classifier-subsystem 32 includes a classifier-subsystem preprocessor 36, a classifier-subsystem feature extractor 38 and the AHP classifier 14. In one embodiment, the classifier-subsystem preprocessor 36 receives the temporal input signals 34 from the second data repository 30. Furthermore, the classifier-subsystem preprocessor 36 processes the temporal input signals 34 to reduce noisy observations from the temporal input signals 34 resulting in generation of preprocessed temporal input signals 40. The classifier-subsystem preprocessor 36, for example, processes the temporal input signals 34 by identifying and selecting the noisy observations in the temporal input signals 34. The noisy observations, for example, may include missing data values, extreme data values, spike data values, or combinations thereof in the temporal input signals 34. It is noted that extreme data values and spike data values may be defined or identified differently for different hemodynamic parameters. For example, for a temporal input signal corresponding to a hemodynamic parameter namely heart rate (HR), an extreme data value may be defined as a heart rate value that is below 5 mmHg. In one embodiment, spike data values in a temporal input signal corresponding to a hemodynamic parameter may be identified based upon a first derivative of the temporal input signal and one or more determined thresholds. Particularly, the spike data values may be determined by identifying derived data values in the first derivative that cross one or more determined thresholds, wherein the spike data values are data values that correspond to the identified derived data values. Furthermore, the classifier-subsystem preprocessor 36 replaces the missing data values by linearly interpolated values. The linearly interpolated values, for example, are determined by determining linear interpolation of the temporal input signals 34. In certain embodiments, when the temporal input signals 34 do not have substantial noisy observations, the classifier-subsystem preprocessor 36 may not process the temporal input signals 34. The classifier-subsystem preprocessor 36, for example, may be a module, executable instructions, a filtering device, or combinations thereof.

Furthermore, as previously noted, the classifier-subsystem 32 further includes the classifier-subsystem feature extractor 38. In one embodiment, the classifier-subsystem feature extractor 38 receives the temporal input signals 34 from the second data repository 30. In the presently contemplated configuration, the classifier-subsystem feature extractor 38 receives the preprocessed temporal input signals 40 from the classifier-subsystem preprocessor 36.

The classifier-subsystem feature extractor 38 determines one or more temporal input features 42 corresponding to one or more of the hemodynamic parameters of the admitted-patient 16. The classifier-subsystem feature extractor 38, for example, may be executable instructions, a module or a processing subsystem/device that includes the executable instructions to perform the functions of classifier-subsystem feature extractor 38. In one embodiment, the classifier-subsystem feature extractor 38 determines the temporal input features 42 corresponding to each of the hemodynamic parameters of the admitted-patient 16. The classifier-subsystem feature extractor 38, for example, determines the temporal input features 42 based upon the temporal input signals 34 or the preprocessed temporal input signals 40. In the presently contemplated configuration, the classifier-subsystem feature extractor 38 determines the temporal input features 42 based upon the preprocessed temporal input signals 40. The temporal input features 42, for example, may include covariance between two or more of the temporal input signals 34/preprocessed temporal input signals 40. The temporal input features 42, for example, may further include a mean of temporal input signals/preprocessed temporal input signals, a median of temporal input signals/preprocessed temporal input signals, a maximum decrement in the expanse of temporal input signals/preprocessed temporal input signals, a maximum increment in the expanse of temporal input signals/preprocessed temporal input signals, a maximum slope of a linear regression of temporal input signals/preprocessed temporal input signals, a minimum slope of a linear regression of temporal input signals/preprocessed temporal input signals, or combinations thereof. Exemplary temporal input features corresponding to a hemodynamic parameter namely ‘heart rate’ is shown with reference to FIG. 4. Similarly, temporal input features corresponding to a hemodynamic parameter namely ‘Systolic Arterial Blood Pressure’ is shown with reference to FIG. 4. The relationship of temporal input signals or preprocessed temporal input signals and temporal input features, for example, may be shown by the following:

Itᵢ=yᵢ (1)

Itᵢ^{HR},Itᵢ^{SABP},Itᵢ^{DABP},Itᵢ^{ABPmean}→yᵢ (2)

wherein Itᵢ represents temporal input signals representative of a plurality of hemodynamic parameters, Itᵢ^{HR} are temporal input signals representative of heart rate, Itᵢ^{SABP} are temporal input signals representative of systolic arterial blood pressure, Itᵢ^{DABP} are temporal input signals representative of diastolic arterial blood pressure, Itᵢ^{ABPmean} are temporal input signals representative of mean ambulatory blood pressure, and where yᵢεR^{d} is a multivariate feature vector that represents the temporal input features 42.

As previously noted, the classifier-subsystem 32 further includes the AHP classifier 14. The AHP classifier 14, for example, is a model, a hyper plane, or both. The AHP classifier 14 receives the temporal input features 42 from the classifier-subsystem feature extractor 38. The AHP classifier 14 predicts potential AHE in the admitted-patient 16 based upon the temporal input features 42. The prediction of the potential AHE in the admitted-patient 16, for example, may include a positive potential AHE in the admitted-patient 16 or a negative potential AHE in the admitted-patient 16. The positive potential AHE shows that the admitted-patient 16 will experience a potential AHE in the next few hours. Similarly the negative potential AHE shows that the admitted-patient 16 will not experience an AHE in the next few hours. The prediction of the potential AHE in the admitted-patient 16 provides reasonable time to practitioners to determine the cause of the potential AHE in the admitted-patient 16. Furthermore, the prediction of the potential AHE in the admitted-patient 16 enables the practitioners to administer appropriate medical aid to prevent the admitted-patient 16 from experiencing the potential AHE.

In certain embodiments, post the prediction of the potential AHE in the admitted-patient 16, the temporal input signals 34 or the preprocessed temporal input signals 40 may be transmitted to the first data repository 18. The first data repository 18 stores the temporal input signals 34 or the preprocessed temporal input signals 40 as temporal training signals to update the temporal training signals 18 resulting in updated temporal training signals (not shown in FIG. 1). Accordingly, the updated temporal training signals include the temporal training signals 18 and the temporal input signals 34 or the preprocessed temporal input signals 40. The processing subsystem 12 may update the acute hypotension prediction classifier 14 based upon the updated temporal training signals.

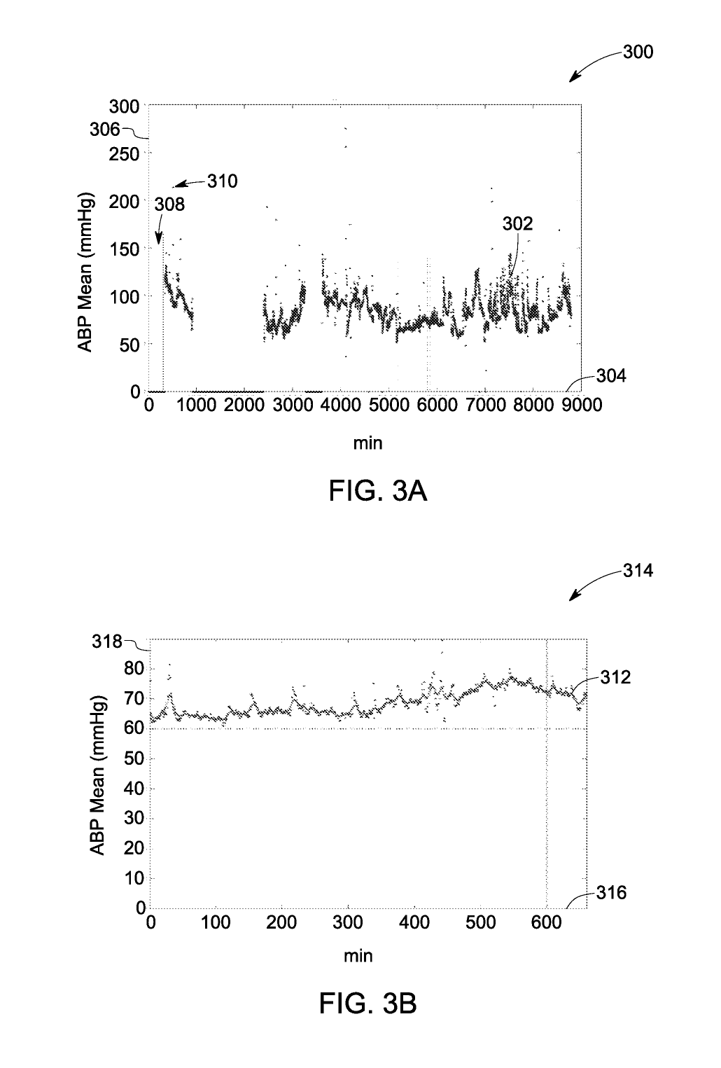

FIG. 2 is a diagrammatic illustration of the architecture of the processing-subsystem 12 referred to in FIG. 1, in accordance with one embodiment of the present systems. The processing subsystem 12 is configured to generate the AHP classifier 14 referred to in FIG. 1. In one embodiment, the processing subsystem 12 includes a processing-subsystem preprocessor 100 that receives and processes the temporal training signals 20, 22 referred to in FIG. 1 to generate preprocessed temporal training signals 102. The processing-subsystem preprocessor 100, for example, may be a module, executable instructions, a filtering device, or combinations thereof. The processing subsystem preprocessor 100, for example, processes the temporal training signals 20, 22 by identifying and selecting noisy observations in the temporal training signals 20, 22. The noisy observations, for example, may include missing data values, extreme data values, spike data values, or combinations thereof in the temporal training signals 20, 22. It is noted that extreme data values and spike data values may be defined or identified differently for different hemodynamic parameters. For example, for a temporal training signal corresponding to a hemodynamic parameter namely heart rate (HR), an extreme data value may be defined as a heart rate value that is below 5 mmHg. In one embodiment, spike data values in a temporal training signal TTS corresponding to a hemodynamic parameter may be identified based upon a first derivative of the temporal training signal TTS and one or more determined thresholds. Particularly, the spike data values may be determined by identifying derived data values in the first derivative that cross the one or more determined thresholds, wherein the spike data values are data values that correspond to the identified derived data values. Furthermore, the processing-subsystem preprocessor 100 replaces the missing data values by linearly interpolated values. The linearly interpolated values, for example, are determined by determining linear interpolation of the temporal training signals 20, 22. An exemplary temporal training signal and a corresponding preprocessed temporal training signal corresponding to a heart associated parameter is shown with reference to FIG. 3a and FIG. 3b, respectively.

The processing subsystem 12 further includes a classifier feature extractor 104. The classifier feature extractor 104 receives the preprocessed training signals 102 from the processing-subsystem preprocessor 100. In one embodiment, the classifier feature extractor 104 determines the temporal training features 28 (referred to in FIG. 1) corresponding to the hemodynamic parameters of the elected-patients 20, 22. The classifier feature extractor 104, for example, determines the temporal training features 28 based upon the preprocessed temporal training signals 102. It is noted that while in the presently contemplated configuration, the classifier-feature extractor 104 determines the temporal training features 28 based upon the preprocessed temporal training signals 102, in certain embodiments, the classifier feature extractor 104 may determine the temporal training features 28 based upon the temporal training signals 20, 22. As previously noted, the temporal training features 28, for example, may include covariance between two or more of the temporal training signals 20, 22 corresponding to at least one of the hemodynamic parameters. The temporal training features 28, for example, may further include a mean of the temporal training signals 20, 22, a median of the temporal training signals 20, 22, a maximum decrement in the expanse of the temporal training signals 20, 22, a maximum increment in the expanse of the temporal training signals 20, 22, a maximum slope of a linear regression of the temporal training signals 20, 22, a minimum slope of a linear regression of the temporal training signals 20, 22, or combinations thereof. Exemplary temporal training features corresponding to a temporal signal representative of hemodynamic parameters namely: Heart Rate and Systolic Blood Pressure is shown in FIG. 4. The relationship of temporal training signals or preprocessed temporal training signals and temporal training features, for example, may be shown by the following:

Ttᵢ=xᵢ (3)

Ttᵢ^{HR},Ttᵢ^{SABP},Ttᵢ^{DABP},Ttᵢ^{ABPmean}→xᵢ (4)

wherein Ttᵢ represents temporal training signals representative of a plurality of hemodynamic parameters, Ttᵢ^{HR} are temporal training signals representative of heart rate Ttᵢ^{SABP} are temporal training signals representative of systolic arterial blood pressure, Ttᵢ^{DABP} are temporal training signals representative of diastolic arterial blood pressure, Ttᵢ^{ABPmean} are temporal signals representative of mean ambulatory blood pressure, and where xᵢεR^{d} is a multivariate feature vector that represents the temporal training features 28.

Furthermore, the processing subsystem 12 includes a classifier generator 106 that receives the temporal training features 28 from the classifier feature extractor 104. The classifier generator 106, for example, may be executable instructions, a processing device configured to run the executable instructions, a module or, the like. Subsequent to the determination of the temporal training features 28, the temporal training features 28 are transmitted to the classifier generator 106. The classifier generator 106 receives the temporal training features 28 from the classifier feature extractor 104. The classifier generator 106 generates the AHP classifier 14 referred to in FIG. 1 based upon the temporal training features 28. Particularly, the classifier generator 106 generates the AHP classifier 14 by applying a support vector machine technique to the temporal training features 28. In one embodiment, the support vector machine technique is a linear support vector machine technique. The AHP classifier 14, for example, may be a hyper plane, a model, or both that is capable of predicting potential acute hypotensive episode in a patient, such as, the admitted patient 16. Particularly, as previously noted with reference to FIG. 1, the AHP classifier 14 receives the temporal input features 42 to predict acute HPE in the admitted-patient 16.

Referring now to FIGS. 3a and 3b, an exemplary graphical representation (hereinafter ‘graph’) 300 of a temporal signal 302 corresponding to a hemodynamic parameter is shown. In one embodiment, the temporal signal 302 may be the temporal training signals 20, 22 (see FIG. 1) or the temporal input signals 34 (see FIG. 1). In this embodiment, the hemodynamic parameter is Mean Ambulatory Blood Pressure (MABP). Accordingly, in this embodiment, the temporal signal 300 represents time-series measurements of the MABP. X-axis 304 of the graph 300 represents time in minutes, and Y-axis 306 of the graph 300 represents mean Ambulatory Blood Pressure Mean (MABP). The temporal signal 302 is sampled for 9000 minutes. The temporal signal 302 contains missing data values, spike data values, outlier data values, extreme data values, and noise. For example, among other missing data values, the temporal signal 302 contains missing data values 308 since time zero till about 600 minutes. Similarly, among other spike data values, the temporal signal 302 contains a spike data value 310. The temporal signal 302, is processed to generate a preprocessed signal. A portion 312 of the pre-processed signal is shown in FIG. 3b via a graphical representation 314. X-axis 316 of the graph 314 represents time in minutes, and Y-axis 318 of the graph shows ABP mean of the portion 312 of the preprocessed signal. In one embodiment, the portion 312 of the preprocessed signal may be the preprocessed temporal training signals 102 or the preprocessed temporal input signals 40 referred to in the FIG. 2 and FIG. 1, respectively. The portion 312 of the preprocessed signal shows a processed temporal signal since zero to about 800 minutes. As shown in the portion 312, in this embodiment, the missing data values 308 in the temporal signal 302 are replaced by linearly interpolated values. Furthermore, the spike data value 310 in the temporal signal 302 does not exist in the portion 312 of the preprocessed signal.

FIG. 4 is an exemplary block diagram that shows features corresponding to temporal signals representative of a hemodynamic parameter namely ‘heart rate’ and ‘Systolic arterial blood pressure.’ As shown in FIG. 4, reference numeral 402 represents temporal signals representative of a heart associated parameter namely ‘Heart Rate’ of a patient. Furthermore, reference numeral 404 represents temporal signals representative of a heart associated parameter namely: ‘Systolic Arterial Blood Pressure’ of the patient. The patient, for example, may be the elected-patients 24, 26 or the admitted-patient 16. The temporal signals 402, for example, may be the temporal training signals 20, 22, the preprocessed temporal training signals 102, the temporal input signals 34 or the preprocessed temporal input signals 40 referred to in FIG. 1.

Reference numeral 406, 408 are representative of features corresponding to the temporal signals 402, 404, respectively. The features 406, 408, for example, may be the temporal training features 28 or the temporal input features 42 referred to in FIG. 1. In this embodiment, the features 406 corresponding to the temporal signals 402 representative of heart rate includes mean heart rate, median heart rate, max decrement of heart rate, maximum increment of heart rate, maximum slope of heart rate, minimum slope of heart rate and co-variance of heart rate. Reference numeral 408 is representative of features corresponding to the temporal signals 404 representative of Systolic Arterial Blood Pressure. In this embodiment, the temporal training features 408 corresponding to the temporal signals 404 includes mean systolic arterial blood pressure (SABP), median SABP, max decrement of SABP, maximum increment of SABP, maximum slope of SABP, minimum slope of SABP and co-variance SABP.

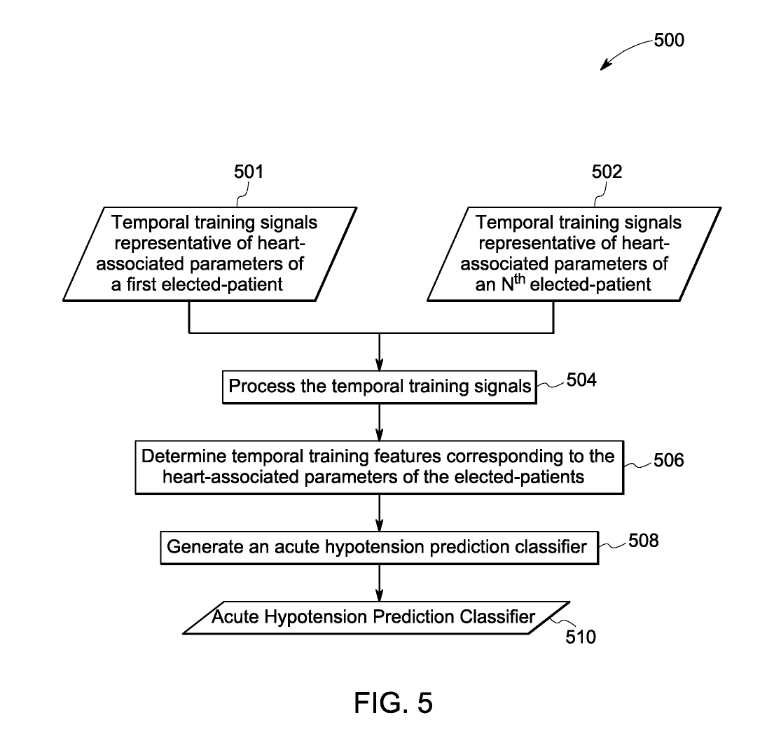

Referring now to FIG. 5, a flow chart illustrating an exemplary method 500 for generating an acute hypotension prediction classifier 510 is shown. Reference numeral 501 is representative of temporal training signals representative of one or more hemodynamic parameters of a first elected patient. Reference numeral 502 is representative of one or more hemodynamic parameters of an N^{h} elected-patient. The temporal training signals 501, 502, for example, may be the temporal training signals 20, 22 referred to in FIG. 1. The temporal training signals 501, 502 are time-series measurements of one or more hemodynamic parameters of the first elected-patient and the n^{th} elected-patient, respectively. It is noted that a single temporal training signal may represent a single hemodynamic parameter of an elected-patient for a determined time period. For example, a temporal training signal S may represent ‘heart rate’ of an elected-patient for 5 hours. The temporal training signals 502, for example may be received from the records of a hospital, research institute, or other source. In one embodiment, the temporal training signals 501, 502 may be time-series signals representative of hemodynamic parameters that are recorded for a determined time period. In one embodiment, the temporal training signals 501, 502 may be records generated for a determined time period after the admission of the 1^{st} and the N^{th} elected-patients in ICU/s and before the beginning of the acute hypotension episode prediction window. For example, the temporal training signals 501, 502 may be records of 10 hours collected during a time period after the admission of the elected-patients and before the beginning of an acute hypotension prediction window which starts at a time T₀.

At step 504, the temporal training signals 501, 502 may be processed to remove noisy observations from the temporal training signals 501, 502. As previously noted, the noisy observations, for example may include missing data values, extreme data values, spike data values, or combinations thereof in the temporal training signals 501, 502. In the presently contemplated configuration, at step 506, a plurality of temporal training features corresponding to the hemodynamic parameters is determined. The temporal training features, for example, may be the temporal training features 28 referred to in FIG. 1. Subsequently at step 508, an acute hypotension prediction classifier 510 is generated based upon the temporal training features. For example, a linear support vector machine technique is applied to the temporal training features to generate the AHP classifier 510. The AHP classifier 510, for example, may be the AHP classifier 14.

The cardiovascular system is a closed hydraulic circuit that includes the heart, arteries, arterioles, capillaries, and veins. Each of the segments of this circuit plays a role in the overall operation of the cardiovascular system in accordance with anatomical volume, resistance to floe, and compliance that are dynamic. In order to capture the dynamic (time-varying) nature of the cardiovascular system for classification and prediction of conditions impacting the operation of the overall cardiovascular system, it is critical to design and extract features that are temporal and indicate trends over time while capturing the dynamics of the cardiovascular system. Accordingly, the present systems and methods design and extract temporal training features and temporal input features that indicate trends over time while capturing the dynamics of the cardiovascular system. The present systems and methods capture the dynamic (time-varying) nature of cardiovascular system of patients for classification and prediction of conditions impacting the operation of the overall system to predict the potential AHE.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method, comprising:
determining a plurality of temporal training features corresponding to one or more hemodynamic parameters of a plurality of elected-patients based upon temporal training signals representative of the one or more hemodynamic parameters; and
generating an acute hypotension prediction classifier based upon the plurality of temporal training features corresponding to the one or more hemodynamic parameters of the plurality of elected-patients,
wherein the plurality of temporal training features comprises covariance between two or more of the temporal training signals corresponding to the one or more hemodynamic parameters.

2. The method of claim 1, further comprising receiving the temporal training signals representative of the one or more hemodynamic parameters of the plurality of elected-patients.

3. The method of claim 1, further comprising processing the temporal training signals to remove noisy observations from the temporal training signals.

4. The method of claim 1, wherein generating the acute hypotension prediction classifier comprises applying a support vector machine technique to the plurality of temporal training features.

5. The method of claim 1, wherein the one or more hemodynamic parameters comprise heart rate (HR), arterial blood pressure, diastolic arterial blood pressure (DABP), systolic arterial blood pressure (SABP), mean ambulatory blood pressure (MABP), or combinations thereof.

6. The method of claim 1, wherein the plurality of temporal training features comprises a mean of the temporal training signals, a median of the temporal training signals, a maximum decrement in the expanse of the temporal training signals, a maximum increment in the expanse of the temporal training signals, a maximum slope of a linear regression of the temporal training signals, a minimum slope of a linear regression of the temporal training signals, or combinations thereof.

7. The method of claim 1, wherein the the plurality of elected-patients comprises patients who had acute hypotensive episodes in the past and patients who did not have acute hypotensive episodes in the past.

8. The method of claim 1, further comprising predicting an event of acute hypotensive episode in an admitted-patient based upon temporal input signals representative of one or more hemodynamic parameters of the admitted-patient.

9. The method of claim 8, wherein predicting the event of acute hypotensive episode comprises:
determining a plurality of temporal input features corresponding to the one or more hemodynamic parameters of the admitted-patient based upon the temporal input signals representative of the one or more hemodynamic parameters; and
predicting the event of acute hypotensive episode in the admitted-patient based upon the plurality of temporal input features and the acute hypotension prediction classifier.

10. The method of claim 8, wherein the event of acute hypotensive episode comprises a positive acute hypotensive episode or a negative acute hypotensive episode in the admitted-patient.

11. The method of claim 8, wherein the temporal input signals comprises time-series measurements generated for a determined time period starting after the admission of the admitted-patient in an intensive care unit till starting of an acute hypotension prediction window.

12. A system, comprising a processing subsystem configured to:
determine a plurality of temporal training features corresponding to one or more hemodynamic parameters of a plurality of elected-patients based upon temporal training signals representative of the one or more hemodynamic parameters; and
generate an acute hypotension prediction classifier based upon the plurality of temporal training features corresponding to the one or more hemodynamic parameters of the plurality of elected-patients,
wherein the plurality of temporal training features comprises covariance between two or more of the temporal training signals corresponding to the at least one hemodynamic parameter.

13. The system of claim 12, wherein the one or more hemodynamic parameters comprise heart rate (HR), arterial blood pressure, diastolic arterial blood pressure (DABP), systolic arterial blood pressure (SABP), mean ambulatory blood pressure mean (MABP), or combinations thereof.

14. The system of claim 12, wherein the plurality of temporal training features comprises a mean of the temporal training signals, a median of the temporal training signals, a maximum decrement in the expanse of the temporal training signals, a maximum increment in the expanse of the temporal training signals, a maximum slope of a linear regression of the temporal training signals, a minimum slope of a linear regression of the temporal training signals, or combinations thereof.

15. The system of claim 12, wherein the acute hypotension prediction classifier is configured to predict an event of acute hypotensive episode in an admitted-patient based upon a plurality of temporal input signals representative of one or more hemodynamic parameters of the admitted-patient.

16. The system of claim 12, further comprising a first data repository that stores the temporal training signals representative of the one or more hemodynamic parameters of the plurality of elected patients.

17. A system, comprising:
a classifier-subsystem feature extractor configured to determine one or more temporal input features corresponding to one or more hemodynamic parameters of an admitted-patient based upon temporal input signals representative of the one or more hemodynamic parameters; and
an acute hypotension prediction classifier configured to predict an event of acute hypotensive episode in the admitted-patient based upon the one or more temporal input features,
wherein the one or more temporal input features comprises covariance between two or more of the temporal input signals representative of the one or more hemodynamic parameters.

18. The system of claim 17, wherein the one or more temporal input features comprises a mean of the temporal input signals, a median of the temporal input signals, a maximum decrement in the expanse of the temporal input signals, a maximum increment in the expanse of the temporal input signals, a maximum slope of a linear regression of the temporal input signals, a minimum slope of a linear regression of the temporal input signals, or combinations thereof.

19. The system of claim 17, further comprising a classifier-subsystem preprocessor that processes the temporal input signals to remove noisy observations from the temporal input signals.

20. A system, comprising a processing subsystem, the processing subsystem comprising:
a processing subsystem preprocessor that processes temporal training signals representative of one or more hemodynamic parameters of a plurality of elected-patients to generate preprocessed temporal training signals;
a classifier feature extractor that determines a plurality of temporal training features corresponding to the one or more hemodynamic parameters of the plurality of elected-patients based upon the preprocessed temporal training signals; and
a classifier generator that generates an acute hypotension prediction classifier based upon the plurality of temporal training features corresponding to the one or more hemodynamic parameters of the plurality of elected-patients,
wherein the plurality of temporal training features comprises covariance between two or more of the temporal training signals corresponding to the one or more hemodynamic parameters.
